(19) United States
(12) Patent Application Publication
(10) Pub. No.: US 2017/0311776 A1
(43) Pub. Date: Nov. 2, 2017

(54) OPTICAL SCANNING APPARATUS

(71) Applicant: OLYMPUS CORPORATION, Tokyo (JP)

(72) Inventor: Atsuyoshi SHIMAMOTO, Tokyo (JP)

(73) Assignee: OLYMPUS CORPORATION, Tokyo (JP)

(21) Appl. No.: 15/654,759

(22) Filed: Jul. 20, 2017

Related U.S. Application Data
(63) Continuation of application No. PCT/JP2015/000305, filed on Jan. 23, 2015.

(51) Int. Cl. A61B 1/00; A61B 1/07; G02B 26/10

(52) U.S. Cl. CPC A61B 1/00013; G02B 26/10; A61B 1/00009; A61B 1/00172; A61B 1/07

(57) ABSTRACT

An optical scanning endoscope apparatus includes: an illumination optical fiber that emits light from a tip part oscillatably supported; an actuator that drives the tip part of the illumination optical fiber; and a signal generator that generates, with respect to the actuator, a drive signal for causing the tip part of the illumination optical fiber to spiral scan. The signal generator generates the drive signal which includes: an amplitude expansion period for expanding the amplitude of the drive signal of the fiber from substantially 0 to a maximum value; and an amplitude contraction period for contracting the amplitude of the drive signal from the maximum value to substantially 0, the drive signal having an envelope that smoothly continues, with a gradient of substantially 0, across a border between the periods, with the longer one of the periods being defined as an effective scanning period.

### CROSS-REFERENCE TO RELATED APPLICATION

The present application is a Continuing Application based on International Application PCT/JP2015/000305 filed on Jan. 23, 2015, the content of which is incorporated herein by reference.

### TECHNICAL FIELD

The present disclosure relates to an optical scanning apparatus that causes a fiber tip to spiral scan.

### BACKGROUND

Apparatuses such as an optical scanning endoscope have been proposed as an apparatus for scanning an object with laser light (see, for example, PTL 1 to 3). Such apparatus irradiates laser light from an oscillatable fiber tip onto an observation object, and vibrates the fiber to sequentially scan the laser light on the observation object to convert transmitted light, reflected light, or fluorescence from the observation object into electric signals by a photoelectric conversion means, to thereby generate an image.

A so-called spiral scan has been adopted as a scheme for driving a fiber of an optical scanning apparatus. The scheme involves rotating the fiber tip while gradually expanding and contracting the amplitude of the fiber (that is, the radius of rotation) between 0 and a maximum value, to thereby scan a predetermined region of a scanning object. As means for scanning a fiber of an optical scanning apparatus, there may be employed, for example, a system of vibrating piezoelectric elements attached to the fiber, or an electromagnetic coil system which uses an electromagnetic coil to vibrate a permanent magnet attached to the fiber. In either case, the driving means is configured to generate driving force in two directions orthogonal to the optical axis of the fiber. Driver elements such as piezoelectric elements and electromagnetic coils may be vibratorily driven at or near the resonance frequency of the fiber to be oscillated, to thereby obtain a large amount of deflection (displacement, amplitude) of the fiber with a small amount of energy.

### CITATION LIST

### Patent Literature

PTL 1: JP5190267B

PTL 2: JP1672023B

PTL 3: JP2014145941A

### SUMMARY

An optical scanning apparatus disclosed herein includes:

a fiber that emits light from a tip part oscillatably supported;

an actuator that drives the tip part of the fiber; and

a signal generator that generates, with respect to the actuator, a drive signal for causing the tip part of the fiber to spiral scan, in which:

the signal generator generates, during one scanning period, a drive signal having a first period and a second period different in length from the first period, the first period expanding the amplitude of the drive signal of the fiber from substantially 0 to a maximum value, the second period contracting the amplitude of the drive signal from the maximum value to substantially 0;

the drive signal has an envelope that smoothly continues, with a gradient of substantially 0, across a border between the first period and the second period; and

a longer one of the first period and the second period is defined as an effective scanning period. Here, in the subject application, the effective scanning period refers to a period that contributes to image generation.

The tip part of the fiber may preferably be driven at a drive frequency different from a resonance frequency.

Further, the envelope of the drive signal in the first period and the envelope of the drive signal in the second period may be configured to constitute part of sinusoidal waveforms mutually different in cycle.

Preferably, the number of laps n₁ in a spiral scan of the fiber in the first period and the number of laps n₂ in a spiral scan of the fiber in the second period are adapted to satisfy:

0.1≦n₁/n₁+n₂≦0.9 (1)

Further, the number of laps n₁ and the number of laps n₂ may be adapted to satisfy, with fᵣ representing a frame rate of the spiral scan:

$$\begin{matrix}{f}_{r} \geq 25 & ( 2 ) \\ 0.2 \leq \frac{{n}_{1}}{{n}_{1} + {n}_{2}} \leq 0.8 . & ( 3 )\end{matrix}$$

Further, the number of laps n₁ and the number of laps n₂ may be adapted to satisfy, with fᵣ representing a frame rate of the spiral scan:

$$\begin{matrix}{f}_{r} \geq 60 & ( 4 ) \\ 0.4 \leq \frac{{n}_{1}}{{n}_{1} + {n}_{2}} \leq 0.6 . & ( 5 )\end{matrix}$$

Further, the disclosed optical scanning apparatus may preferably satisfy:

$$\begin{matrix}{f}_{m 1} = \frac{{f}_{d}}{2 {n}_{1}} \\ and & ( 6 ) \\ \frac{1}{2 {f}_{m 2}} \leq \frac{1}{{f}_{r}} - \frac{{n}_{1}}{{f}_{d}} ; \\ or & ( 7 ) \\ {f}_{m 2} = \frac{{f}_{d}}{2 {n}_{2}} \\ and & ( 8 ) \\ \frac{1}{2 {f}_{m 1}} \leq \frac{1}{{f}_{r}} - \frac{{n}_{2}}{{f}_{d}} , & ( 9 )\end{matrix}$$

where fₘ₁ represents a first modulation frequency as a frequency of amplitude modulation of the first period, and f ₘ₂ represents a second modulation frequency as a frequency of amplitude modulation of the second period, with f_{d} representing a drive frequency of the fiber, ᵣ representing a frame rate of the spiral scan, n₁ representing the number of laps of the spiral scan of the fiber during the first period; n₂ representing the number of laps of the spiral scan of the fiber during the second period.

The disclosed optical scanning apparatus may include:

a light detector for detecting light obtained from an object irradiated with the illumination light; and

an image generator generating an image based on a signal detected by the light detector during the effective scanning period.

In this case, the image generator may suitably generate an image in a shorter one of the first period and the second period.

Further, the irradiation of the illumination of light may preferably be stopped in a shorter one of the first period and the second period.

### BRIEF DESCRIPTION OF THE DRAWINGS

In the accompanying drawings:

FIG. 1 is a block diagram illustrating a schematic configuration of an optical scanning endoscope apparatus as an example of the disclosed optical scanning apparatus according to Embodiment 1,

FIG. 2 is an overview schematically illustrating the scope of FIG. 1;

FIG. 3 is a sectional view of the tip part of the scope of FIG. 2;

FIG. 4A is a sectional view showing the actuator and the oscillating part of the illumination optical fiber of FIG. 3;

FIG. 4B is a sectional view taken along the line A-A of FIG. 4A;

FIG. 5 shows, in a simplified manner, a drive signal of one scan by the signal generator;

FIG. 6A shows an example of a drive signal envelope in triangle waves;

FIG. 6B shows an example of a drive signal envelope in sinusoidal waves;

FIG. 7A shows a fiber scanning locus when the drive signal having the envelope of FIG. 6A is input;

FIG. 7B shows a fiber scanning locus when the drive signal having the envelope of FIG. 6B is input;

FIG. 8 shows a scanning path of the fiber;

FIG. 9A shows an example of a scanning locus of the fiber in one frame;

FIG. 9B shows an enlarged scanning locus near the minimum value of the amplitude of FIG. 9A;

FIG. 10 shows an exemplary case where image information is missing at the image center;

FIG. 11 shows a change of amplitude convergence rate varies relative to the number of laps ratio and the frequency ratio by simulation;

FIG. 12A shows a result obtained by the simulation of FIG. 11 at a frame rate of 15 fps;

FIG. 12B shows a result obtained by the simulation of FIG. 11 at a frame rate of 25 fps;

FIG. 12C shows a result obtained by the simulation of FIG. 11 at a frame rate of 60 fps;

FIG. 13A shows an envelope of the fiber vibration locus at a frame rate of 15 fps, with the number of laps ratio of the drive waveform being changed from 0.1 to 0.9;

FIG. 13B shows an envelope of the fiber vibration locus at a frame rate of 25 fps, with the number of laps ratio of the drive waveform being varied from 0.1 to 0.9;

FIG. 13C shows an envelope of the fiber vibration locus at the frame rate of 60 fps, with the number of laps ratio of the drive waveform being changed from 0.1 to 0.9;

FIG. 14 shows simulation results of the fiber amplitude convergence is rate when the vibration Q value of the fiber is varied from 50 to 400;

FIG. 15 shows simulation results obtained for the amplitude convergence rate of a fiber by changing the resonance frequency of the fiber from 8500 Hz to 9500

FIG. 16 shows in a simplified manner another example of the drive signal of one scan obtained by the signal generator 16; and.

FIG. 17 shows a drive signal in a conventional spiral scan.

### DETAILED DESCRIPTION

When the fiber is driven in practice by spiral scan near the resonance frequency, there arises a phenomenon where the vibration has trouble converging even if the once-enlarged fiber amplitude is tried to be reduced to 0. For example, when the drive signal is stopped in order to naturally attenuate the vibration of the fiber after the amplitude of spiral scan has reached its maximum value, the vibration slowly attenuates at or near the resonance frequency thereof. Suppose the drive signal is applied to enlarge the amplitude again without waiting the vibration of the fiber to be attenuated to 0, the object will then remain unscanned in a region corresponding to the scan center of the fiber. Accordingly, in the case of a scanning endoscope, an increase in frame rate may cause a phenomenon where an image in the screen center cannot be obtained.

In view of the above, after the amplitude has reached the maximum value from 0 during spiral scan, a drive signal shifted in phase by 180° (that is, the drive signal in the reverse direction) from that used during the amplitude expansion can be applied so as to apply so-called “brakes” to the vibration of the fiber, to thereby rapidly attenuate the vibration of the fiber. However, in optical scanning apparatuses, conditions to attenuate vibration of the fiber could sensitively vary depending on changes in properties (such as, for example, the resonance frequency and the Q value) to be generated due to environmental changes. This makes it hard to control the attenuation of the fiber vibration. For example, when the environmental temperature varies, the resonance frequency of the fiber may be shifted, which could hinder the amplitude to converge to 0. In practice, we have simulated a case where the resonance frequency has been deviated by 10 Hz, to find that the vibration of the fiber is not completely attenuated, leaving a minute vibration still lasted for a certain period of time. Here, it may be conceivable to provide the scanning apparatus with a sensor to monitor the vibration frequency of the fiber and to apply a drive signal in the reverse direction according to the actual vibration. However, such configuration will increase the size of the tip part of the scanning apparatus, which is particularly undesirable in the case of an endoscope apparatus.

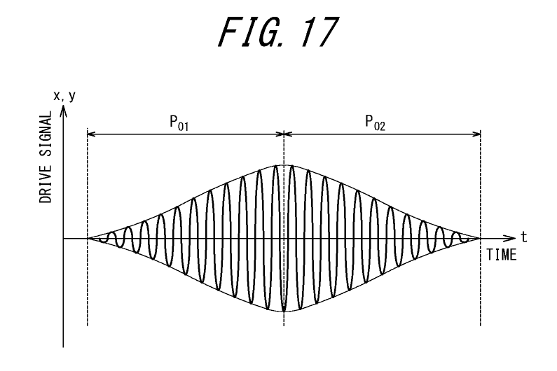

In light thereof, there is proposed a scanning endoscope apparatus, in which, with the drive frequency being largely different from the resonance frequency, the amplitude modulation waveform of the drive signal is sinusoidally deformed so as to converge the fiber vibration to the scan center during the amplitude contraction, to thereby prevent occurrence of voids in the image. FIG. 17 shows an example of a drive signal during one expansion/contraction of the amplitude. This amplitude expansion/contraction is repeatedly performed when actually scanning the fiber. This configuration allows the vibration amplitude of the optical fiber to follow up the drive signal so as to converge to 0 more rapidly as compared with the case of natural attenuation, which can repeat the expansion and contraction of the amplitude at shorter cycles.

However, when this method is used to display a moving image by acquiring images alternately in an amplitude expansion period P₀₁ and an amplitude reduction period P₀₂, there is a fear that the images be slightly deviated or distorted for each one frame because the spiral scan path during the amplitude expansion (hereinafter, also referred to as “outward path”) and the spiral scan path during the amplitude contraction (hereinafter, also referred to as “return path) on the observation object are different from each other in a strict sense. Meanwhile, when an image is acquired in either one of the outward path and the return path to display a moving image, an effective scanning period to be used for image generation is reduced to half, in one of the amplitude expansion and reduction period of the fiber. Thus, in the case of a scanning endoscope apparatus, the number of laps of the fiber scan for use in image generation decreases, and the resolution may likely to fall to one-half.

Hereinafter, an embodiment of the present disclosure will be illustrated with reference to the accompanying drawings.

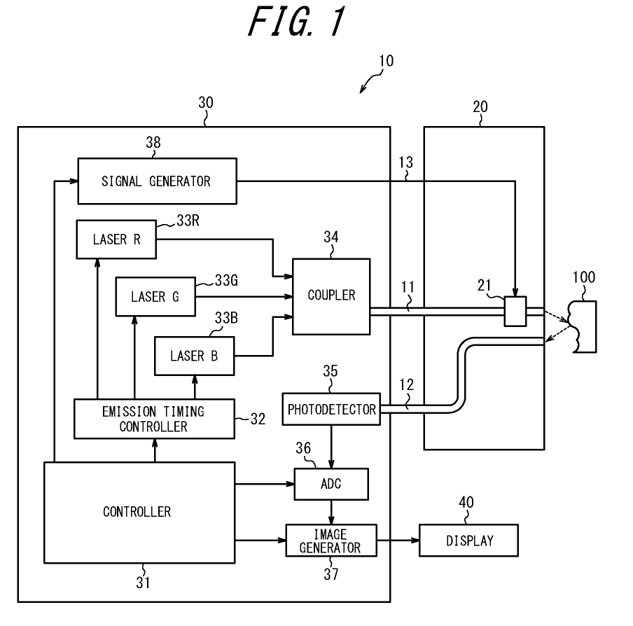

FIG. 1 is a block diagram showing a schematic configuration of an optical scanning endoscope as an example of the disclosed optical scanning apparatus. In FIG. 1, the optical scanning endoscope apparatus 10 includes: a scope 20; a controller body 30; and a display 40.

First, the configuration of the controller body 30 is explained. The controller body 30 includes: a controller 31 controlling the whole of the optical scanning endoscope apparatus 10; an emission timing controller 32; lasers 33R, 33G, 33B; a coupler 34, a photodetector 35 (light detector); an analog-digital converter (ADC) 36; an image generator 37; and a signal generator 38.

The emission timing controller 32 controls the emission timings of the lasers 33R, 33G, 33B each emitting laser lights of red (R), green (G), and blue (B), respectively, according to the control signal from the controller 31. The lasers are controlled so as to emit respective colors of light at every fixed time intervals in an emission order (of, for example, R, G, B, G) determined based on the set values of the emission frequency ratio (of, for example, 1:2:1 in order of R, G. B).

The lasers 33R, 33G, 33B constitute a light source which selectively emits a plurality of different colors (three colors of R, G, and B in this embodiment) of light. The lasers 33R, 33G, 33B may use, for example, a diode pumped solid state laser (DPSS laser) or a laser diode.

Laser light emitted from each of the lasers 33R, 33G, 33B travels through an optical path coaxially combined by the coupler 34, to be incident as illumination light on an illumination optical fiber 11 (fiber) configured as a single mode fiber. The coupler 34 is configured by using, for example, a dichroic prism. The lasers 33R, 33G, 33B and the coupler 34 may be accommodated in a separate casing different from the controller body 30, the casing being connected with the controller body 30 via a signal line.

Light incident from the coupler 34 onto the illumination optical fiber 11 is guided to the tip part of the scope 20, and irradiated therefrom toward an object 100. In doing so, the signal generator 38 of the controller body 30 vibratorily drives an actuator 21 of the scope 20, to thereby vibratorily drive the tip part of the illumination optical fiber 11. This way allows the illumination light emitted from the illumination optical fiber 11 to two-dimensionally scan the observation surface of the object 100. The object 100 thus irradiated with the illumination light provides light such as reflected light and scattered light, which are received by the tip of a detection optical fiber 12 formed of a multimode fiber and guided through inside the scope 20 up to the controller body 30.

The photodetector 35 detects, for each emission cycle of the light source, light obtained via the detection optical fiber 12 from the object 100 irradiated with light of either one of the colors of R, G, B, and outputs an analog signal (electric signal).

The ADC 36 converts the analog signal from the photodetector 35 into a digital signal (electric signal), and outputs the resulting signal to the image generator 37.

The image generator 37 sequentially stores, in a memory (not shown), the digital signals which are input from the ADC 36 for each emission cycle and corresponds to the respective colors, in association with the emission timing and the scanned position, respectively. The information on the emission timing and the scanned position is obtained from the controller 31. The controller 31 calculates information on the scanned position on the scanning path, based on information on the amplitude and phase of an oscillation voltage applied by the signal generator 38 etc. Alternatively, the controller 31 may hold in advance, as a table, information on the scanned position on the scanning path corresponding to the time elapsed from the start of driving. Then, the image generator 37 performs, after the scan or during the scan, necessary image processing such as enhancement processing, γ processing, and interpolation processing, based on the digital signal input from the ADC 36, to generate an image signal and displays an image of the object 100 on the display 40.

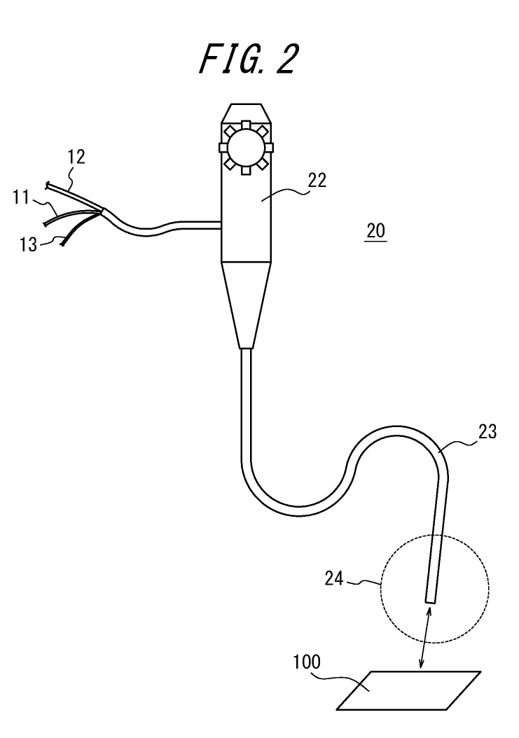

Next, the configuration of the scope 20 is described. FIG. 2 is an overview schematically illustrating the scope 20. The scope 20 includes an operation portion 22 and an insertion portion 23. The operation portion 22 is connected with each of the illumination optical fiber 11, a plurality of the detection optical fibers 12, and a wiring cable 13 from the controller body 30. The illumination optical fiber 11, the detection optical fibers 12, and the wiring cable 13 run through inside the insertion portion 23 to be extended up to the tip part 24 (portion within the broken line of FIG. 2) of the insertion portion 23.

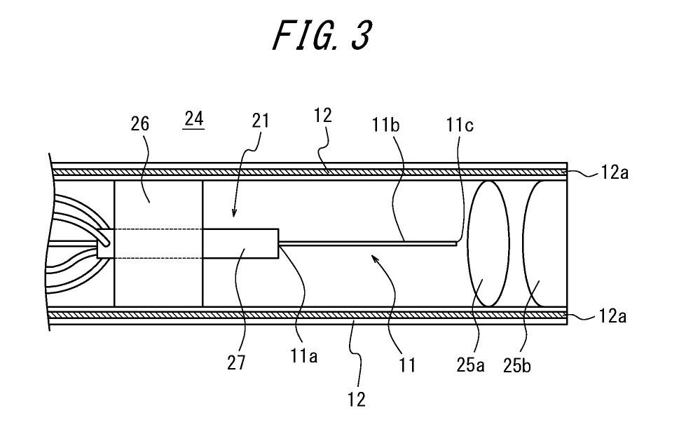

FIG. 3 is an enlarged sectional view of the tip part 24 of the insertion portion 23 of the scope of FIG. 2. The tip part 24 of the insertion portion 23 of the scope 20 is configured by including the actuator 21, projection lenses 25a, 25b, the illumination optical fiber 11 passing through the center, and a plurality of the detection optical fibers 12 passing through the outer periphery.

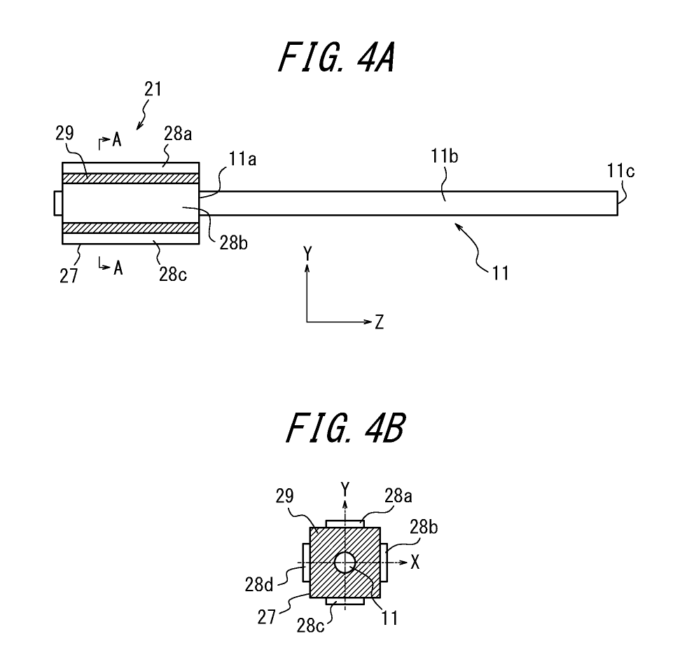

The actuator 21 vibratorily drives the tip part 11c of the illumination optical fiber 11. The actuator 21 is configured by including an actuator tube 27 fixed inside the insertion portion 23 of the scope 20 by means of an attachment ring 26, and a fiber holding member 29 and piezoelectric elements 28a to 28d each disposed inside the actuator tube 27 (see FIGS. 4A and 4B). The illumination optical fiber 11 is supported by a fiber supporting member 29, with a part defined from a fixed end 11a supported by the fiber holding member 29 to the tip part 11c serving as an oscillation part 11b oscillatably supported. Meanwhile, the detection optical fibers 12 are disposed to pass through the outer periphery of the insertion portion 23 and extended to the leading end of the tip part 24. Further, the detection optical fibers 12 may each include, at the tip part thereof, a detection lens, which is not shown.

Further, the projection lenses 25a, 25b and the detection lens are disposed at the extreme tip of the tip part 24 of the insertion portion 23 of the scope 20. The projection lenses 25a, 25b are configured such that the laser light emitted from the tip part 11c of the illumination optical fiber 11 is irradiated onto the object 100 as being substantially converged. The detection lens is disposed to take in light resulting from the laser light that has been converged onto the object 100 and reflected, scattered, and refracted by the object 100, so as to have it converged and coupled to the plurality of the detection optical fibers 12 disposed behind the detection lens. Here, the projection lenses may include a single lens or a plurality of lenses other than two, without being limited to the two-lens configuration.

FIG. 4A illustrates a vibration driving mechanism of the actuator 21 and the oscillation part 11b of the illumination optical fiber 11 of the optical scanning endoscope apparatus 10, and FIG. 4B is a sectional view taken along the line A-A of FIG. 4A. The illumination optical fiber 11 penetrates the center of the fiber holding member 29 in a rectangular prism shape, so as to be fixedly held by the fiber holding member 29. The four sides of the fiber holding member 29 each face either in the ±Y-directions and ±X-directions. Then, the piezoelectric elements 28a, 28c with the same expansion and contraction properties for Y-direction driving, are disposed in a pair on both sides in the ±Y-directions of the fiber holding member 29, while the piezoelectric elements 28b, 28d with the same expansion and contraction properties for X-direction driving, are disposed in a pair on both sides in the ±X-directions of the fiber holding member 29.

The piezoelectric elements 28a to 28d are each connected with the wiring cable 13 from the signal generator 38 of the controller body 30, and driven through application of a voltage by the signal generator 38.

Voltages applied across the piezoelectric elements 28b and 28d in the X-direction are always the same in magnitude and opposite in polarity. Similarly, voltages applied across the piezoelectric elements 28a and 28c in the Y-direction are always the same in magnitude and opposite in polarity. Of the piezoelectric elements 28b, 28d disposed as opposing to each other across the fiber holding member 29, one expands while the other contracts in a reciprocal manner, to thereby cause deflection in the fiber holding member 29, which may be repeated to generate vibration in the X-direction. Vibration in the Y-direction may similarly be caused.

The signal generator 38 applies vibration voltages which have the same amplitude and gradually expand and contract while shifted in phase by 90 degrees to the piezoelectric elements 28b, 28d for X-direction driving and the piezoelectric elements 28a, 28c in the Y-direction driving, to thereby vibratorily drive the piezoelectric elements 28a, 28c for Y-direction driving and the piezoelectric elements 28b, 28d for X-direction driving. In this manner, the oscillation part 11b of the illumination optical fiber 11 of FIGS. 3, 4A, and 4B vibrates, and the tip part 11c is deflected so as to draw a spiral locus, with the result that the laser light emitted from the tip part 11c spirally scans in sequence the surface of the object 100.

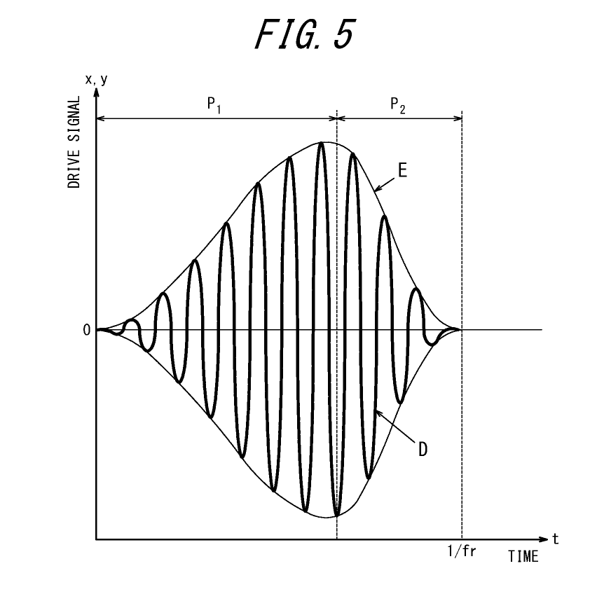

FIG. 5 shows in a simplified manner a drive signal of one scan by the signal generator 38. Here, one scan means a cycle of scan performed when the amplitude of a drive signal expands from 0 to the maximum value and then contracts from the maximum value to 0, which corresponds to one frame of image acquisition by the optical scanning endoscope apparatus 10. FIG. 5 shows a curve of a drive signal D, with a signal value (driving voltage) of the drive signal in x-, y-directions being on the ordinate and the time being on the abscissa. The drive signals in the x-direction and y-direction are shifted in phase by 90 degrees, but the difference is not shown. The drive signal D is a signal obtained from a vibration voltage modulated in amplitude, with the envelope E thereof exhibiting a modulation waveform. Here, FIG. 5 shows the drive signal D in a simplified manner for the sake of explanation, and thus, the actual period of the drive signal is far shorter than one frame. For example, the frequency of one frame is on the order of several tens of Hz, while the driving frequency may be defined on the order of several hundreds to thousands of Hz. Here in the disclosure, when the amplitude of the drive signal or the inclination of the modulation waveform are defined as 0, it should include a range that can be identified as 0 (substantially 0) within error margin.

The envelope E or the modulation waveform of the drive signal of the signal generator 38 of this embodiment smoothly continues across the border between an amplitude expansion period P₁ (first period) and an amplitude contraction period P₂ (second period), with a gradient of substantially 0. This allows the amplitude of the fiber scan to decrease following the drive signal D in the amplitude contraction period P₂. It is noteworthy that the present disclosure is configured to contract the amplitude the drive voltage within the shorter period, i.e., the amplitude contraction period P₂, rather than turning off the drive signal D, after the amplitude of the drive signal D has reached the maximum value. In this manner, the amplitude can be converged to 0 more rapidly as compared with the case of turning off the signal to let the amplitude of the illumination optical fiber 11 naturally attenuated. As a result, a stable scan can be achieved with no void in the center. Here, an envelope that “smoothly continues with a gradient of substantially 0” means that the envelope (modulation waveform) continues across the border between the amplitude expansion period P₁ and the amplitude contraction period P₂, always with a differential value of 0.

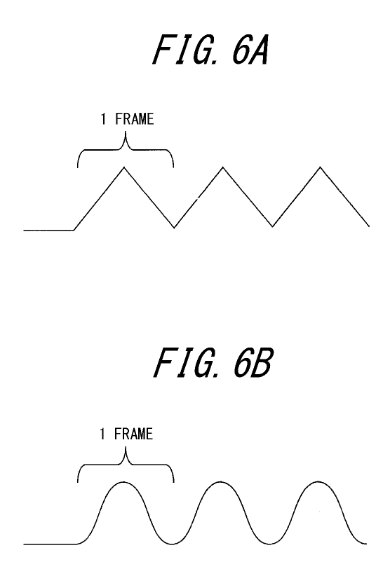

Described is an effect to be obtained when the envelope continues smoothly with a gradient of substantially 0. FIGS. 6A and 6B show exemplary waveforms of the drive signal envelope. FIG. 6A shows triangle waves, which do not continue smoothly. On the other hand, FIG. 6B shows sinusoidal waves by way of example, which continue smoothly. The drive signal in either case is formed by alternately repeating the amplitude expansion period and the amplitude contraction period.

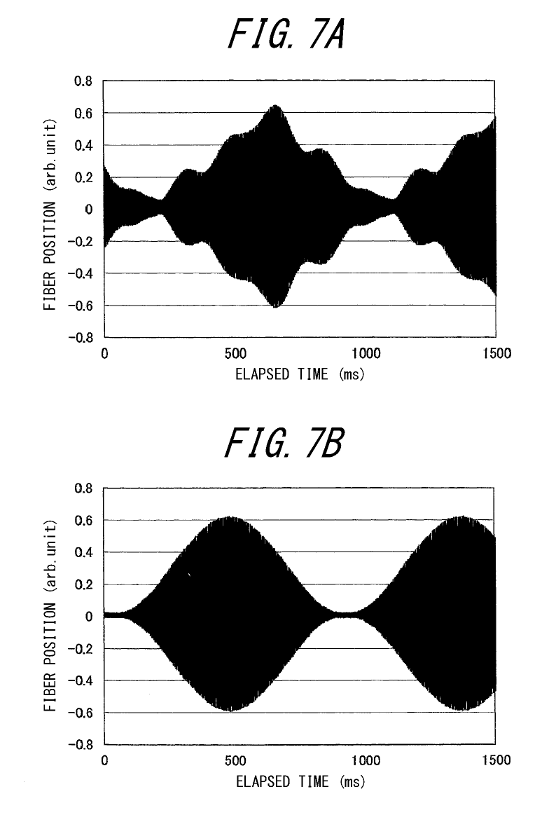

FIGS. 7A and 7B each show a response of the fiber scanning locus to the input of each of the drive signals of FIGS. 6A and 6B. FIG. 7A corresponds to the case of the envelope in triangle waves and FIG. 7B corresponds to the case of the envelope in sinusoidal waves. In the case of FIG. 7A, the gradient do not smoothly continue, which applies a rapid change of acceleration to the fiber across the border between the amplitude expansion period and the amplitude contraction, making the vibration unstable. On the other hand, in the case of FIG. 7B, where the gradient smoothly continues, no rapid change of acceleration is applied to the fiber across the border between the amplitude expansion period and the amplitude contraction, which stabilizes the vibration. Therefore, the gradient may desirably continue smoothly with a gradient of substantially 0.

Further, in FIG. 5, when the amplitude expansion period P₁ and the amplitude contraction period P₂ are compared with each other, the amplitude expansion period P₁ is longer than the amplitude contraction period P₂. Then, in this embodiment, the image generator 37 generates an image based on image signals obtained by the photodetector 35 during the amplitude expansion period P₁. Accordingly, in this case, the amplitude expansion period P₁ serves as the effective scanning period that contributes to image generation.

Further, image signals obtained by the photodetector 35 in the amplitude expansion period P₁ may be processed by the image generator 37 in the amplitude contraction period P₂. This way allows the throughput of the controller body 30 to be temporally distributed, to thereby attain efficient processing in the device as a whole.

In particular, in this embodiment, the envelope E or the amplitude modulation waveform constitutes part of sinusoidal waves different from one another in modulation frequency in each of the amplitude expansion period P₁ and the amplitude contraction period P₂. For example, when the amplitude expansion period P₁ has a modulation frequency of fₘ₁ and the amplitude contraction period P₂ has a modulation frequency of fₘ₂, fₘ₁ is given by the following expression (6) (as already mentioned above)

$${f}_{m 1} = \frac{{f}_{d}}{2 {n}_{1}} \qquad ( 6 )$$

In the expression (6), f_{d} represents a drive frequency of the drive signal n₁ represents a desired number of laps of the tip part 11c of the illumination optical fiber 11 in the amplitude expansion period P₁.

Further, fₘ₂ is defined to satisfy the following inequality (7) (as already mentioned above).

$$\frac{1}{2 {f}_{m 2}} \leq \frac{1}{{f}_{r}} - \frac{{n}_{1}}{{f}_{d}} \qquad ( 7 )$$

In the inequality (7), fᵣ represents a frame rate. As can be seen therefrom, the amplitude modulation waveform may be defined as a sinusoidal waveform without including unnecessary frequency components, to thereby alleviate image distortion during the amplitude expansion period P₁ while rapidly returning the tip part 11c of the illumination optical fiber 11 to the scan center during the amplitude contraction period P₂, that is, during when no image is being generated, which can reduce voids in the image to be generated.

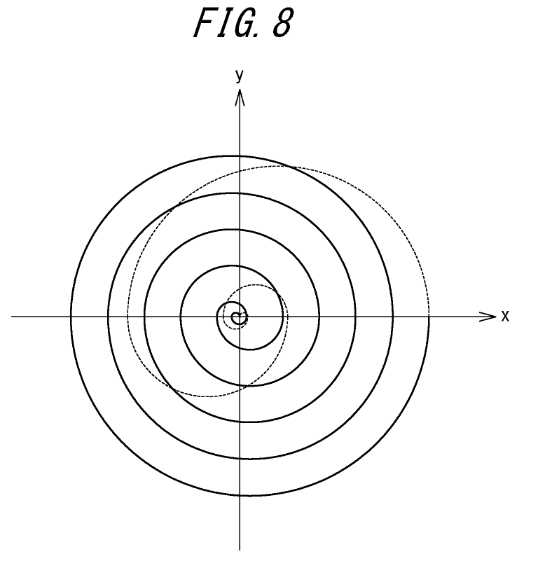

FIG. 8 shows images of scanning paths on the object 100 rendered by the illumination optical fiber 11. The solid lines shows the scanning path during the amplitude expansion period P₁, and the broken line shows the scanning path during the amplitude contraction period P₂. The optical scanning endoscope apparatus 10 expands the amplitude from the scan center by drawing a spiral and obtains an image signal during the amplitude expansion period P₁, and when the maximum value of the scanning amplitude has been reached, more rapidly reduces the amplitude toward the scan center during the amplitude contraction period P₂. Here, FIG. 8 is for explanation purpose only; it should be noted that the number of laps of the actual scanning waveform is much larger and the scanning density in the radial direction is far higher than those of FIG. 8.

As described above, the amplitude expansion period P₁ contributing image generation is longer than the amplitude contraction period P₂. Accordingly, with respect to one entire scanning period, the ratio of the amplitude contraction period P₂ not being used for image generation is relatively small, and thus causes no significant loss of the effective scanning period. Accordingly, the number of laps of the tip part 11c of the illumination optical fiber 11 during the effective scanning period can be increased, which can enhance the resolution of the optical scanning endoscope apparatus 10.

Further, the drive frequency of the drive signal generated by the signal generator 38 may preferably set to a value largely different from the resonance frequency of the oscillation part 11b of the illumination optical fiber 11, so as to more rapidly reduce the amplitude during the amplitude contraction period P₂.

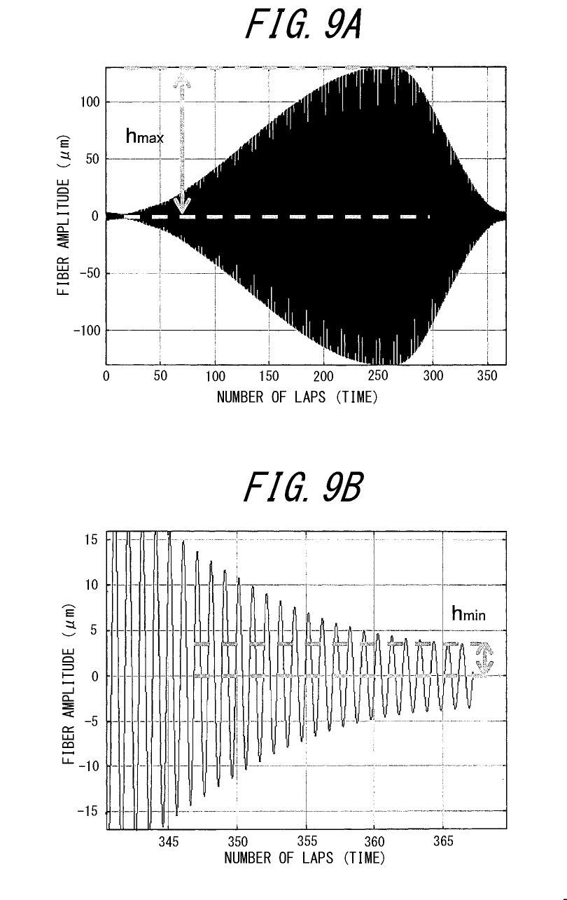

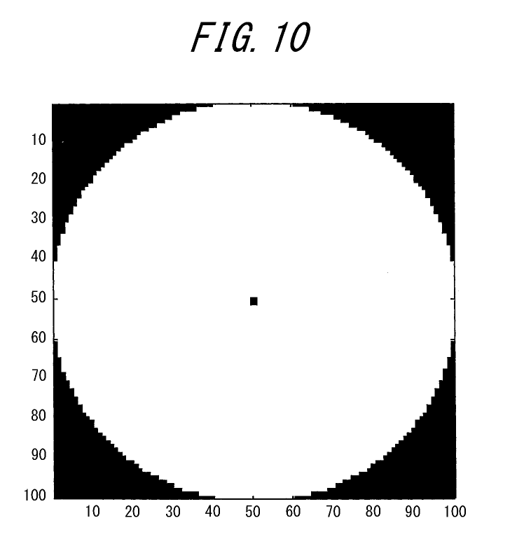

Here, consideration is given of how much of the amplitude of the fiber should be returned to the scan center in order not to greatly affect the image quality of the image center. FIG. 9A shows an example of the scanning locus of the fiber in one frame. Further, FIG. 9B is an enlarged view of the scanning locus near the minimum value of the amplitude. The maximum value and the minimum value of the fiber amplitude in one frame are each defined as hmax and hmin, respectively, and the amplitude convergence rate is defined as hmin÷hmax×100[%]. The maximum radius of the illumination region onto the object is associated with the maximum value hmax of the amplitude. On the other hand, when the fiber is not attenuated to 0, the center of the illumination region goes missing, generating an unilluminated region. The radius of the region is associated with the minimum value hmin of the amplitude. Once missing occurs, the pixel information at the image center is lost as illustrated in FIG. 10 in the case where the image is generated based on the scanning locus position measured in advance by a measuring instrument such as a position sensor device (PSD). Here, the white portion shows pixels where the locus passes through, and the black portion shows pixels where the locus does not pass through. The image of FIG. 10 contains, for example, 100×100 pixels and has the amplitude convergence rate of about 5%.

In order to obtain excellent effect in terms of resolution as compared with a fiber scanning endoscope which employs an image light guide using a bundle fiber that can similarly be reduced in diameter, the displayed image may desirably be equivalent to 100×100 pixels or more. In an image of 100×100 pixels, when the aforementioned amplitude convergence rate is 2%, the pixel information loss at the center is obtained as 100×0.02=2 pixels. The pixel information loss of 2 pixels or less would not significantly affect the sense of resolution, through image processing such as pixel interpolation process. However, the loss of 2 or more pixels should greatly affect the sense of resolution at the image center. Thus, in a fiber scanning endoscope, the amplitude convergence rate may preferably be 2% or less.

Here, the conditions of the waveform of the drive signal for suppressing the amplitude convergence rate to 2% or less may be considered. n₁ represents a desired number of laps of the tip part 11c of the illumination optical fiber 11 during the amplitude expansion period P₁, and n₂ is defined as a desired number of laps of the tip part 11c of the illumination optical fiber 11 during the amplitude contraction period P₂, so as to define the number of laps ratio as n₁/(n₁+n₂). The number of laps ratio takes a value from 0 to 1, and determines the waveform of the envelope of the drive waveform. With the value closer to 0, the drive waveform has an envelope longer in a period on the amplitude contraction side. With the value closer to 1, the drive waveform has an envelope longer in a period on the amplitude expansion side. Further, the drive frequency of the drive signal is defined as f_{d} and the resonance frequency of the vibration of the illumination optical fiber 11 is defined as f_{c}, so as to determine the frequency ratio as f_{d}/f_{c}. The amplitude convergence rate becomes smaller as the drive frequency is deviated farther from the resonance frequency, that is, as the frequency ratio is increased to be larger than 1 or smaller than 1.

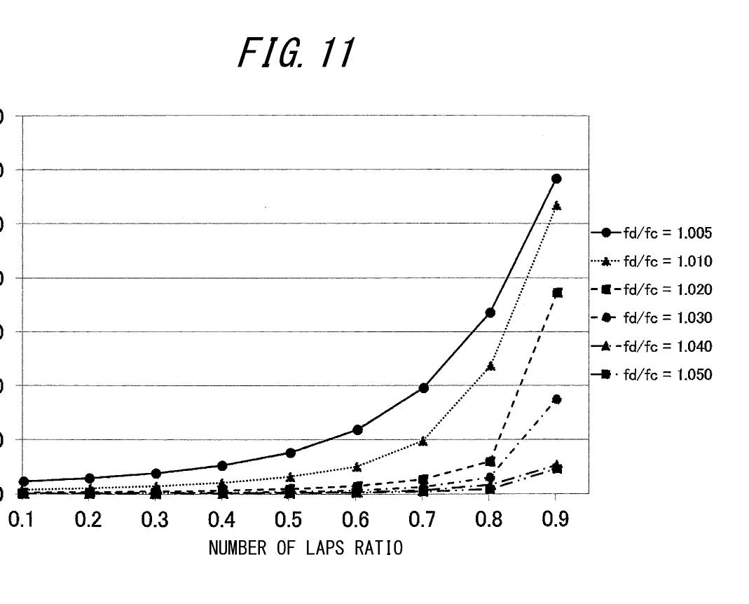

FIG. 11 shows an exemplary result of a simulation obtained for the change in the amplitude convergence ratio by changing the values of the above-defined two parameters, namely, the “number of laps ratio” and the “frequency ratio”. In the simulation, calculation is performed with the vibration Q value of the fiber being 100, the resonance frequency of the fiber being 9000 Hz, and the frame rate being 25 Hz, assuming that the fiber follows damping vibration. Further, the fiber is driven to have a drive frequency larger than the resonance frequency. As can be understood from FIG. 11, the missing of the locus may be reduced, the amplitude convergence rate may be smaller, and the resolution at the center may be improved, as the number of laps ratio is reduced and the frequency ratio is increased to be larger than 1. Although not shown in the drawing, our calculation holds the aforementioned relation, irrespective of the vibration Q value and the resonance frequency of the fiber. Further, although not shown in the drawing, our calculation similarly holds the aforementioned relation as the frequency ratio is further reduced to be smaller than 1.

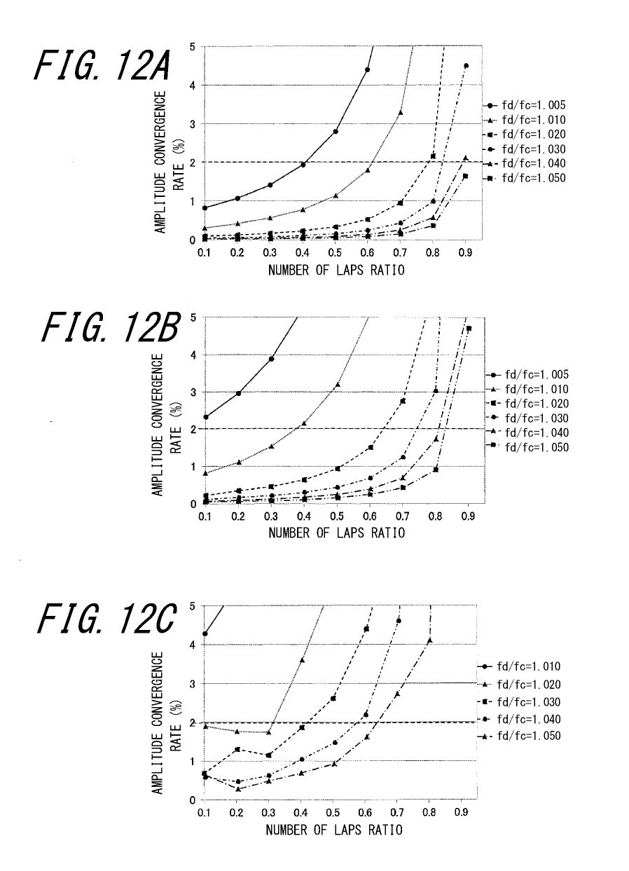

FIGS. 12A, 12B, and 12C show the results obtained from the aforementioned simulation with different frame rates. Here, FIGS. 12A, 12B, and 12C each show the case with the frame rates of 15, 25, and 60, respectively, which all fall under sufficient conditions capable of capturing a moving image. FIGS. 12A, 12B, and 12C show that the amplitude convergence rate increases along with the increase of the frame rate, as long as the frequency ratio and the number of laps ratio are constant. As described above, the amplitude convergence is required to be 2% or less so as not to affect the sense of resolution at the image center. FIG. 12A shows that the conditions may be achieved with the frame rate of 15 and the frequency ratio of 1.05, when the number of laps ratio is 0.9. Therefore, the number of laps ratio may desirably be 0.9 or less.

Further, conditions for satisfying the amplitude convergence rate of 2% or less when the frame rate is 25 or more are examined. It can be understood from FIG. 12B that the number of laps ratio of 0.9 or more requires the frequency ratio to be larger than 1.05, which means that the fiber is driven at a frequency greatly different from the resonance frequency, with the result that the amplitude will significantly decrease. The frequency ratio may be set to 1.04 or more when the number of laps is 0.8, and thus, the number of laps ratio may desirably be 0.8 or less when the frame rate is 25 or more.

Similarly, conditions for satisfying the amplitude convergence rate of 2% or less when the frame rate is 60 or more are examined. It can be understood from FIG. 12C that the number of laps ratio of 0.7 or more requires the frequency ratio to be larger than 1.05, which means that the fiber is driven at a frequency greatly different from the resonance, causing significant reduction in amplitude. The frequency ratio may be set to 1.05 when the number of laps is 0.6, and thus, the number of laps ratio may desirably be 0.6 or less when the frame rate is 60 or more.

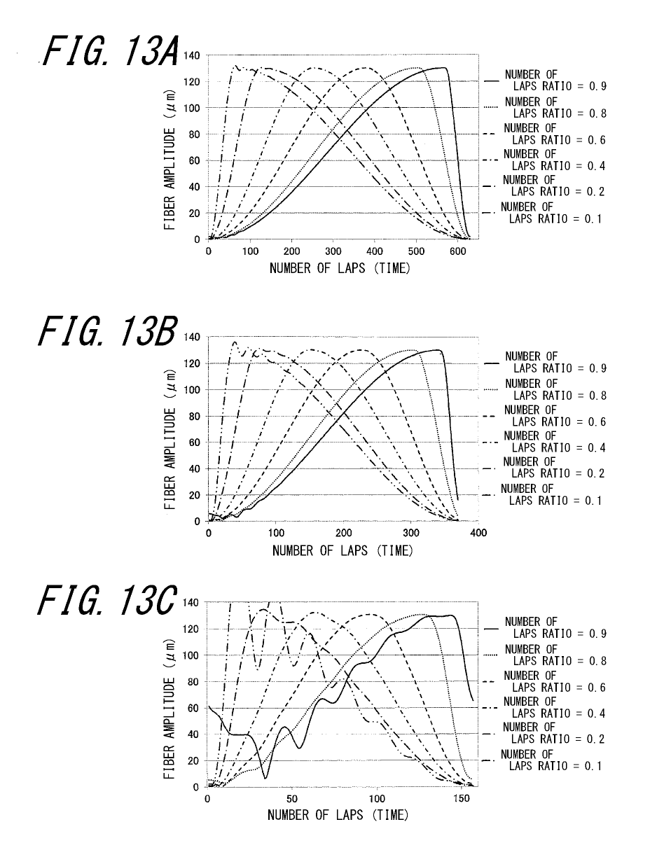

Next, the lower limit conditions of the number of laps ratio are examined. FIGS. 13A, 13B, and 13C each show an envelope of the fiber vibration locus obtained, with the number of laps ratio of the drive waveform being varied from 0.1 to 0.9. The envelope renders a locus for just one frame, and the fiber repeatedly vibrates along the envelope for each frame. FIGS. 13A, 13B, and 13C each show simulation results with the frame rates of 15, 25, and 60, respectively. The simulation results have been obtained by calculation, with the fiber having the vibration Q value of 100, the resonance frequency of 9000 Hz, and the frequency ratio of 1.03, assuming that the fiber follows damping vibration. Further, the fiber is driven to have a drive frequency larger than the resonance frequency.

FIGS. 13A, 13B, and 13C each show that the envelope is waved as the number of laps ratio becomes smaller, which makes the vibration unstable. Further, the envelope thus waved tends to show condensation and rarefaction on the scanning density of the spiral scan, which affects the resolution. FIGS. 13A, 13B, and 13C also show that the envelope curve is waved complexly as the frame rate grows larger, making the vibration further unstable. Accordingly, the number of laps ratio may desirably be larger than a certain value, depending on the frame rate. Although not shown in the drawings, our calculation holds the aforementioned relation, irrespective of the vibration Q value and the resonance frequency value of the fiber.

Specifically, in order to stabilize the envelope curve when the number of laps is 0.1, the frame rate and the frequency ratio may he set to 15 and 0.1, respectively, as can he seen from FIG. 13A. Thus, the number of laps ratio may desirably be 0.1 or more. Further, when the frame rate is 25 or more, the frequency ratio may desirably be set to 0.2 or more to stabilize the envelope curve, as can be seen from FIG. 13B. Similarly, when the frame rate is 60 or more, the frequency ratio may desirably be set to 0.4 or more to stabilize the envelope curve, as can be seen from FIG. 13C.

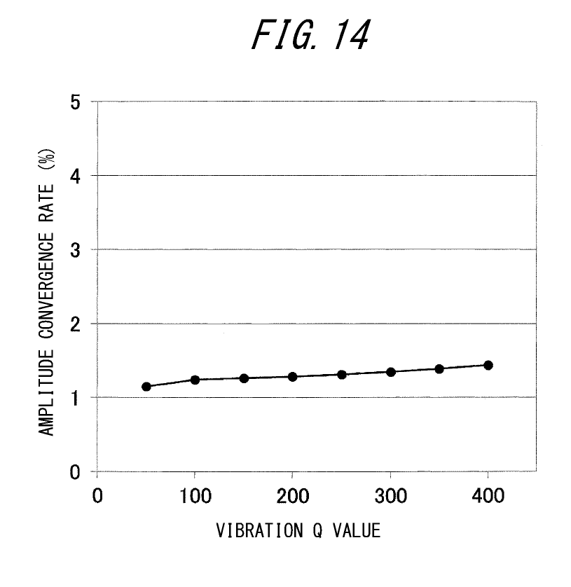

FIG. 14 shows simulation results of the fiber amplitude convergence rate when the vibration Q value of the fiber is varied from 50 to 400. The simulation results have been obtained by calculation with the fiber having the resonance frequency of 9000 Hz, the frame rate of 25 Hz, the frequency ratio of 1.03, and the number of laps ratio of 0.7, assuming that the fiber follows damping vibration. As can be seen from FIG. 14, when the fiber has a vibration Q value falling between about 50 to 400, the parameters of the frequency ratio and the number of laps become dominant as described above, with the amplitude convergence ratio being less affected by the vibration Q value. Although not shown in the drawing, our calculation similarly holds the aforementioned relation even when the frequency ratio is smaller than 1.

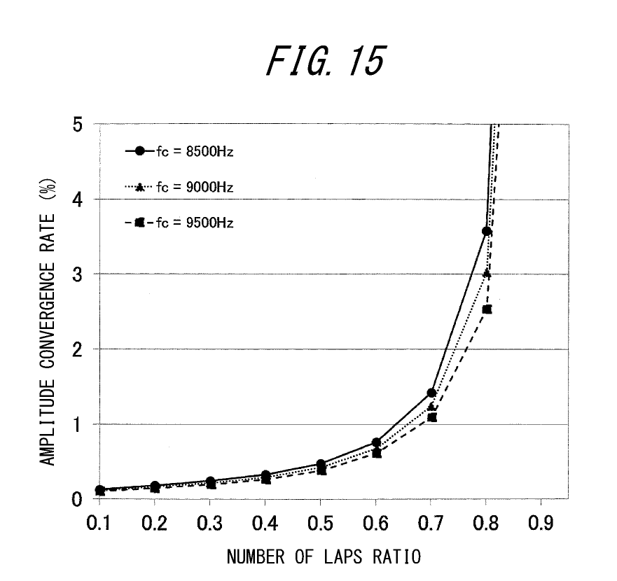

FIG. 15 shows simulation results of the fiber amplitude convergence rate when the resonance frequency of the fiber is varied from 8500 Hz to 9500 Hz. The simulation results have been obtained by calculation, with the fiber having the vibration Q value of 100, the frame rate of 25 Hz, the frequency ratio of 1.03, assuming that the fiber follows damping vibration. As can be seen from FIG. 15, in the amplitude convergence rate, the parameters of the frequency ratio and the number of laps become dominant over the resonance frequency of the fiber as described above. Although not shown in the drawing, our calculation similarly holds the aforementioned relation even when the frequency ratio is smaller than 1.

Here, in the amplitude contraction period P₂, the illumination light is not used for image generation, and thus, the irradiation of the illumination light may be stopped during the period without affecting the image quality. Meanwhile, the amount of laser light per unit time needs to be smaller than the standard value of laser safety. Thus, in view of the above, the illumination light may be irradiated during the amplitude expansion period P₁ while the illumination light may be stopped during the amplitude contraction period P₂, to thereby reduce the total irradiation light amount of the laser per one frame, which can reduce the critical value of laser safety.

As described above, according to this embodiment, the signal generator 38 generates a drive signal including, in one scanning period, the amplitude expansion period P₁ for expanding the amplitude of the drive signal of the illumination optical fiber 11 from 0 to the maximum value and the amplitude contraction period P₂ for reducing the amplitude of the drive signal from the maximum value to 0. The envelope E of the drive signal D smoothly continues, with a gradient of 0, across the border between the amplitude expansion period P₁ and the amplitude contraction period P₂. The amplitude expansion period P₁, which is longer than the amplitude contraction period P₂, is defined as the effective scanning period, and thus causing no significant loss of the effective scanning period, to thereby reduce missing of the scanning paths at the scan center, allowing for stable scan.

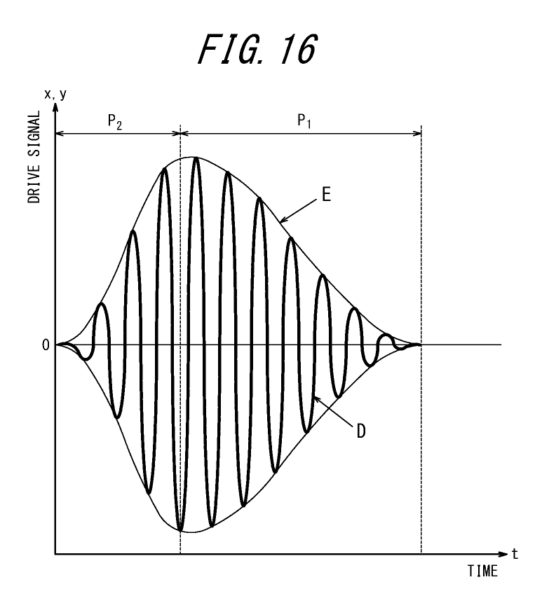

Note that various modifications and alterations are available to of the present disclosure, without being limited only to the aforementioned embodiment. For example, of the amplitude expansion period P₁ and the amplitude contraction period P₂, the effective scanning period is not limited to the amplitude expansion period P₁. For example, the optical scanning endoscope apparatus 10 may be configured to obtain image signals during the contraction of the scanning amplitude. In this case, as illustrated in FIG. 16, the amplitude of the drive signal may be modulated such that the amplitude contraction period P₂ becomes longer than the amplitude expansion period P₁.

An idea similar to that of the expression (6) may be applied to make the amplitude contraction period P₂ longer than the amplitude expansion period P₁, and fₘ₂ may be given by the following expression (8) (as already mentioned above).

$${f}_{m 2} = \frac{{f}_{d}}{2 {n}_{2}} \qquad ( 8 )$$

Here, f_{d} is a drive frequency of the drive signal, n₂ is a desired number of laps of the tip part 11c of the illumination optical fiber 11 during the amplitude contraction period P₂.

Further, fₘ₁ is defined to satisfy the following inequality (9) (as already mentioned above).

$$\frac{1}{2 {f}_{m 1}} \leq \frac{1}{{f}_{r}} - \frac{{n}_{2}}{{f}_{d}} \qquad ( 9 )$$

Here, fᵣ is a frame rate. As can be understood therefrom, the amplitude modulation waveform may be defined as a sinusoidal waveform without including unnecessary frequency components, to thereby reduce image distortion to occur during the amplitude contraction period P₂ while rapidly returning the tip part 11c of the illumination optical fiber 11 to the scan center, which can reduce voids in the image to be generated.

Further, the actuator of the illumination optical fiber of the optical scanning apparatus is not limited to the one using the piezoelectric elements.

For example, an electromagnetic driving method is also available using magnets and coils. In this case, unlike the aforementioned embodiment, where a voltage to be applied to the piezoelectric elements was controlled by a drive signal, the electromagnetic driving method may use a drive signal so as to control a current value to flow through the coils.

Further, the optical scanning apparatus may be applied to a projector and other optical scanning apparatuses, without being limited to an optical scanning endoscope.

### REFERENCE SIGNS LIST

10 optical scanning endoscope apparatus

11 illumination optical fiber

11a fixed end

11b oscillation part

11c tip part

12 detection optical fiber

13 wiring cable

20 scope

21 actuator

22 operation portion

23 insertion portion

24 tip part

25a, 25b projection lens

26 attachment ring

27 actuator tube

28a to 28d piezoelectric elements

29 fiber holding member

30 controller body

31 controller

32 illumination timing controller

33R, 33G, 33B laser

34 coupler

35 photodetector

36 ADC

37 image generator

38 signal generator

40 display

100 object

P₁ amplitude expansion period

P₂ amplitude contraction period

D drive signal

E envelope

1. An optical scanning apparatus, comprising:
a fiber that emits light from a tip part oscillatably supported;
an actuator that drives the tip part of the fiber; and
a signal generator that generates, with respect to the actuator, a drive signal for causing the tip part of the fiber to spiral scan,
wherein:
the signal generator generates a drive signal having, in one scanning period, a first period and a second period different in length from the first period, the first period expanding the amplitude of the drive signal of the fiber from substantially 0 to a maximum value, the second period contracting the amplitude of the drive signal from the maximum value to substantially 0;
the drive signal has an envelope that smoothly continues, with a gradient of substantially 0, across a border between the first period and the second period; and
a longer one of the first period and the second period is defined as an effective scanning period.

2. The optical scanning apparatus according to claim 1, wherein the tip part of the fiber is driven at a drive frequency different from a resonance frequency.

3. The optical scanning period according to claim 1, wherein the envelope of the drive signal in the first period and the envelope of the drive signal in the second period each form part of sinusoidal waves mutually different in cycle.

4. The optical scanning apparatus according to claim 1, satisfying:
$$0.1 \leq \frac{{n}_{1}}{{n}_{1} + {n}_{2}} \leq 0.9 , \qquad ( 1 )$$
where n₁ represents the number of laps in a spiral scan of the fiber during the first period, and n₂ represents the number of laps in a spiral scan of the fiber during the second period.

5. The optical scanning apparatus according to claim 4, wherein the number of laps n₁ and the number of laps n₂ satisfy, with fᵣ representing a frame rate of the spiral scan:
$$\begin{matrix}{f}_{r} \geq 25 ; \\ and & ( 2 ) \\ 0.2 \leq \frac{{n}_{1}}{{n}_{1} + {n}_{2}} \leq 0.8 . & ( 3 )\end{matrix}$$

6. The optical scanning apparatus according to claim 4, wherein the number of laps n₁ and the number of laps n₂ satisfy, with fᵣ representing a frame rate of the spiral scan:
$$\begin{matrix}{f}_{r} \geq 60 : \\ and & ( 4 ) \\ 0.4 \leq \frac{{n}_{1}}{{n}_{1} + {n}_{2}} \leq 0.6 . & ( 5 )\end{matrix}$$

7. The optical scanning apparatus according to claim 3, satisfying:
$$\begin{matrix}{f}_{m 1} = \frac{{f}_{d}}{2 {n}_{1}} \\ and & ( 6 ) \\ \frac{1}{2 {f}_{m 2}} \leq \frac{1}{{f}_{r}} - \frac{{n}_{1}}{{f}_{d}} ; \\ or & ( 7 ) \\ {f}_{m 2} = \frac{{f}_{d}}{2 {n}_{2}} \\ and & ( 8 ) \\ \frac{1}{2 {f}_{m 1}} \leq \frac{1}{{f}_{r}} - \frac{{n}_{2}}{{f}_{d}} , & ( 9 )\end{matrix}$$
where fₘ₁ represents a first modulation frequency as a frequency of amplitude modulation of the first period, and fₘ₂ represents a second modulation frequency as a frequency of amplitude modulation of the second period, with f_{d} representing a drive frequency of the fiber, fᵣ representing a frame rate of the spiral scan, n₁ representing the number of laps of the spiral scan of the fiber during the first period, n₂ representing the number of laps of the spiral scan of the fiber during the second period.

8. The optical scanning apparatus according to claim 1, further comprising:
a light detector for detecting light obtained from an object irradiated with the illumination light; and
an image generator generating an image based on a signal detected by the light detector during the effective scanning period.

9. The optical scanning apparatus according to claim 8, wherein the image generator generates an image in a shorter one of the first period and the second period.

10. The optical scanning apparatus according to claim 1, wherein the irradiation of the illumination light is stopped in a shorter one of the first period and the second period.
